(19) United States
(12) Patent Application Publication
(10) Pub. No.: US 2024/0230611 A1
(43) Pub. Date: Jul. 11, 2024

(54) DEVICE AND METHOD FOR THE MULTISENSORY MEASUREMENT OF ADSORPTION AND DESORPTION OF COMPOUNDS IN A FLUID

(71) Applicant: ARYBALLE, GRENOBLE (FR)

(72) Inventors: Van Tri NGUYEN, GRENOBLE (FR); Yanis CARITU, GRENOBLE (FR)

(21) Appl. No.: 18/559,707

(22) PCT Filed: May 3, 2022
(86) PCT No.: PCT/IB2022/054065

§ 371 Date: Nov. 8, 2023

(30) Foreign Application Priority Data
May 10, 2021 (FR) FR2104909

(51) Int. Cl. G01N 33/00

(52) U.S. Cl. CPC G01N 33/0031

(57) ABSTRACT

An electronic device for multisensory measuring the adsorption and desorption of compounds present in a fluid includes at least one olfactory sensor designed to interact with a plurality of compounds likely to be present in the fluid and provide a plurality of signals representative of a presence of the organic compounds in the fluid. It further includes a processor for processing the provided signals to obtain a characterization of the fluid composition. This processor is programmed to determine the temporal evolution of at least one variable estimated on the basis of a combination of temporal variations of the provided signals and, using this temporal evolution, to provide a temporal labeling indicating a start and an end of at least an adsorption state and a desorption state relative to the provided signals.

The present invention concerns an electronic device for multisensory measurement of adsorption and desorption of compounds present in a gaseous or liquid fluid. It also concerns a method implemented by such a device and a corresponding computer program.

The invention applies more particularly to an electronic device for multisensory measurement of adsorption and desorption of compounds present in a fluid, comprising:
at least one olfactory sensor designed to:
   interact with a plurality of compounds likely to be present in the fluid,
   provide a plurality of signals representative of a presence of these organic compounds in the fluid; and
a processor for processing the provided signals to obtain a characterization of the fluid composition.

it is understood that the interaction with the compounds, the provision of signals and their processing are operations that can be carried out in parallel over time. Alternatively, processing can be carried out a posteriori.

This type of device is sometimes referred to as a multivariate sensor. It can be used for a plurality of olfactory assessment applications, and is generally referred to as an “electronic nose”. It is then used to detect, discriminate and identify volatile organic compounds in a gaseous fluid to be analyzed, or compounds present in a liquid. It can be used in a variety of industrial fields, such as:
the perfume industry, for example to compare, study or design pure or blended olfactory compounds,
environmental protection, in particular for detecting odorous pollution or monitoring the quality of more or less confined environments,
monitoring of industrial sites where there is a risk of contamination by potentially hazardous or odorous volatile or solvent-soluble materials,
health, for example to offer a substitute for the sense of smell to people suffering from anosmia, or to detect volatile biological markers such as the emanations of infectious microbiological activity,
the food industry, for example to detect contamination in a food manufacturing and/or distribution chain,
any other industrial field where control of an odorous product may be useful.

One example is known and described in patent documents WO 2018/158458 A1 and WO 2019/053366 A1. This example, called NeOse Pro (registered trademark), is marketed by Aryballe company since 2018. It is based on surface plasmon resonance detection technology, i.e., an SPR (for Surface Plasmon Resonance) imaging system, and comprises a processor capable of providing an M-component vector digital signature from the electrical signals provided by the olfactory sensor(s) featuring a plurality of reactive sites, each signature being specific to a detected odor. In general, M is less than or equal to the number of reactive sites. By training on a plurality of fluid samples and under different conditions, it is potentially possible to identify all odors.

To ensure the correct operation of such a device, it is generally necessary to rigorously control the moments of contact with the fluid to be analyzed and those of contact with a reference medium. In this way, usable temporal signals called “sensorgrams” are obtained, characteristic of an interaction kinetics between the target compounds in the fluid and the reactive sites of the electronic measuring device, from which it is possible to characterize the composition of the fluid.

In accordance with the teaching of the article by Bernet et al, entitled “Highly-selective optoelectronic nose based on surface plasmon resonance imaging for sensing volatile organic compounds”, published in Analytical Chemistry 2018, 90, 16, pages 9879-9887 (ACS Publications) on Jul. 19, 2018, a suitable measurement protocol consists in controlling the injection and evacuation of the fluid to be analyzed in a measurement chamber of the electronic device where the reactive sites are arranged, in a sufficiently controlled manner so that the interaction kinetics between the target compounds and the receptors of the reactive sites reach an equilibrium steady state from which it is easier to obtain the characterization of the fluid to be analyzed. More precisely, the fluidic supply to the measuring chamber comprises three successive states: a first reference state of exposure of the reactive sites to a reference fluidic environment with carrier fluid without the presence of the target compounds, i.e. without the presence of the fluid to be analyzed, then a second analytical state of exposure of the reactive sites to the fluid to be analyzed by injection of the latter into the measuring chamber, then a third final state, known as desorption, of re-exposure of the reactive sites to the reference fluidic environment by evacuation of the fluid to be analyzed from the measuring chamber. This fluidic supply is advantageously provided by valves with precisely controlled opening and closing, enabling an a priori knowledge of the different states for better control of the equilibrium steady state, during which the characteristics of the fluid to be analyzed are advantageously extracted from the sensorgrams. All these constraints make the electronic measurement device more complex to build and less simple to use. In addition, it takes a relatively long time to reach the equilibrium steady state during the second analytical exposure state. The result is a relatively slow characterization of the fluid to be analyzed.

A solution to reduce these constraints is proposed in patent document WO 2020/141281 A1. This solution consists in analyzing the temporal evolution of normalized values of a vector constituting the sensorgrams. Surprisingly, during the second state of injection of the fluid to be analyzed into the measuring chamber, the sensorgrams formed on the basis of such a normalized vector reach a steady state much more quickly than conventional sensorgrams, and it is in fact possible to characterize the fluid as soon as this steady state of the normalized sensorgrams is reached. The result is faster processing. It has even been observed that normalized sensorgrams can achieve stability even when conventional sensorgrams fail to reach steady state, particularly when the adsorption/desorption equilibrium in the measuring chamber is not under control. But this does not totally eliminate the need for precise control of fluid flows, at least for a priori knowledge of the three aforementioned exposure states.

In addition, even with flow control, it is still quite difficult to know precisely the three exposure states because of unavoidable delays and time lags, which must also be taken into account.

It may therefore be desirable to provide a measuring device that avoids at least some of the above-mentioned problems and constraints.

An electronic device for multisensory measurement of adsorption and desorption of compounds present in a fluid is therefore proposed, comprising:
at least one olfactory sensor designed to:
   interact with a plurality of compounds likely to be present in the fluid,
   provide a plurality of signals representative of a presence of these organic compounds in the fluid; and
a processor for processing the provided signals to obtain a characterization of the fluid composition;
   wherein the processor is programmed to:
determine the temporal evolution of at least one variable estimated on the basis of a combination of temporal variations of the provided signals; and
using this temporal evolution, provide a temporal labeling indicating a start and an end of at least one adsorption state and one desorption state related to the provided signals.

Indeed, without even needing to know a priori the various aforementioned states, it has been noted that these can surprisingly be directly deduced from a study of the temporal evolution of a variable estimated on the basis of a combination of temporal variations of the provided signals, in particular the aforementioned sensorgrams. In this way, it is no longer necessary to constrain oneself to rigorous control and an a priori knowledge of the successive exposures of the measuring device to the reference medium and the fluid to be analyzed. It is therefore possible to dispense with the need to control valves, or even a measuring chamber. This makes the measurement device much simpler to design and use, particularly in detection and measurement conditions that are closer to the reality usually faced by a human nose for example, i.e., on demand or continuously, without knowing in advance the possible presence of an odor to be detected.

Optionally, the processor can be programmed to:
provide the temporal labeling by comparing said at least one estimated variable to at least one threshold; and
compute said at least one threshold on the basis of temporal variations in the provided signals during a reference state of exposure of said at least one olfactory sensor to a reference fluidic environment.

Also optionally, said at least one estimated variable comprises at least one of the elements of the set consisting of:
a temporal standard deviation computed over a sliding window and averaged over the provided signals;
an instantaneous first derivative averaged over the provided signals; and
a temporally averaged first derivative computed over the sliding window and averaged over the provided signals.

Also optionally, the provided signals comprise at least one of the elements of the set consisting of:
a first set of temporal signals, called sensorgrams, characteristic of an interaction kinetics between the fluid compounds and said at least one olfactory sensor; and
a second set of normalized temporal signals, called normalized sensorgrams, obtained from the sensorgrams of the first set and by computing at each measurement or sampling instant a norm of the vector formed by the values assumed by the sensorgrams of the first set at that instant.

A method is also provided for multisensory measurement of adsorption and desorption of compounds present in a fluid, using an electronic device comprising:
at least one olfactory sensor designed to:
   interact with a plurality of compounds likely to be present in the fluid,
   provide a plurality of signals representative of a presence of these organic compounds in the fluid; and
a processor for processing the provided signals to obtain a characterization of the fluid composition;
   the method further including the following steps, performed by executing the processor:
determine the temporal evolution of at least one variable estimated on the basis of a combination of temporal variations of the provided signals;
using this temporal evolution, provide a temporal label indicating a start and an end of at least one adsorption state and one desorption state related to the provided signals.

Optionally, a multisensory measurement method according to the present invention may comprise the provision of a temporal labeling of a reference state of exposure of said at least one olfactory sensor to a reference fluidic environment not containing the compounds whose adsorption and desorption are to be measured, this temporal labeling being defined as follows:
start of reference state at first sample of provided signals; and
end of reference state as soon as a temporal standard deviation computed over a sliding window and averaged over the provided signals is greater than or equal to a reference threshold predefined as being proportional to a maximum reached by this temporal standard deviation over K first samples of the provided signals, with K∈ [symbol] .

Also optionally, the temporal labeling of adsorption and desorption states is defined as follows:
for the adsorption state: by a temporal labeling binary signal indicating the adsorption state by a high binary value, this binary signal resulting from a logical combination:
   of a first intermediate binary signal F_{δ̅} with a high binary value for any sample of the provided signals for which the temporal standard deviation computed over a sliding window and averaged over the provided signals is greater than an adsorption/desorption threshold predefined as being proportional to a maximum reached by this temporal standard deviation during the reference state, and
   of a second intermediate binary signal F_{d̅}^{ad} with a high binary value for any sample of the provided signals for which a first derivative averaged over the provided signals is greater than an adsorption threshold predefined as being proportional to a maximum reached by this first derivative during the reference state;
for the desorption state: by a temporal labeling binary signal indicating the desorption state by a high binary value, this binary signal resulting from a logical combination:
   of the first intermediate binary signal F_{δ̅}, and
   of a third intermediate binary signal F_{d̅}^{des} with a high binary value for any sample of the provided signals for which the first derivative averaged over the provided signals is lower than a desorption threshold predefined as being proportional to a minimum reached by this first derivative during the reference state;
      and wherein the temporal standard deviation and the first derivative are computed on temporal signals, called sensorgrams, characteristic of interaction kinetics between fluid compounds and said at least one olfactory sensor.

Also optionally, a multisensory measurement method according to the present invention may comprise the provision of a temporal labeling of a plateau state defined by a temporal labeling binary signal indicating the plateau state by a high binary value, this binary signal being with a high binary value for any sample of the provided signals for which the temporal standard deviation computed over the sliding window and averaged over the provided signals is less than or equal to a plateau threshold predefined as being proportional to a maximum reached by this temporal standard deviation during the reference state.

Also optionally, a multisensory measurement method according to the present invention may comprise the provision of a temporal labeling of states of higher stability and odor presence defined:
for the state of higher stability: by a temporal labeling binary signal indicating the state of higher stability by a high binary value, this binary signal being of high binary value for any sample of the provided signals for which a complement to 1 of a normalized temporal standard deviation computed over the sliding window and averaged over the provided signals is greater than a threshold of higher stability predefined as being proportional to a maximum reached by this normalized temporal standard deviation during the whole duration of the provided signals; and
for the state of odor presence: by a temporal labeling binary signal indicating the odor presence state by a high binary value, this binary signal being of high binary value for any sample of the provided signals for which the complement to 1 of the normalized temporal standard deviation computed over the sliding window and averaged over the provided signals is greater than an odor presence threshold predefined as being proportional to a maximum reached by this normalized temporal standard deviation during the reference state;
   and the normalized temporal standard deviation is computed on normalized temporal signals, called normalized sensorgrams, obtained from non-normalized temporal signals characteristic of an interaction kinetics between the fluid compounds and said at least one olfactory sensor and by computing at each measurement or sampling instant a norm of the vector formed by the values taken by the non-normalized temporal signals.

Also proposed is a computer program downloadable from a communications network and/or stored on a computer-readable medium and/or executable by a processor, comprising instructions for executing the steps of a multisensory measurement method according to the present invention, when said program is executed on a computer.

The invention will be better understood with the aid of the following description, given solely by way of example and with reference to the appended drawings wherein:

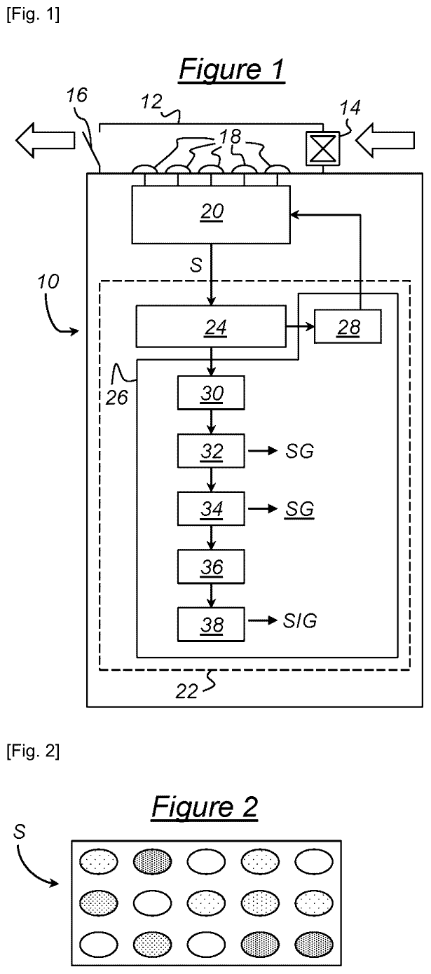

FIG. 1 diagrammatically shows the general structure of an electronic device for multisensory measurement of adsorption and desorption of compounds present in a fluid, according to one embodiment of the invention,

FIG. 2 illustrates a typical schematic example of an image produced by the electronic device shown in FIG. 1, wherein the device's olfactory sensors are visible,

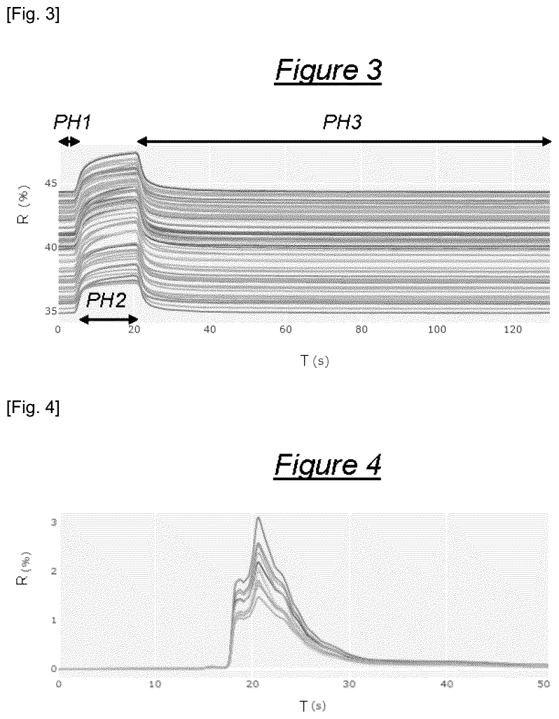

FIG. 3 illustrates a first example of the superposition of response signal temporal diagrams, or sensorgrams, that can be obtained from the image in FIG. 2, under rigorously controlled multisensory measurement conditions,

FIG. 4 illustrates a second example of the superposition of response signal temporal diagrams, or sensorgrams, that can be obtained from the image in FIG. 2, under less controlled multisensory measurement conditions,

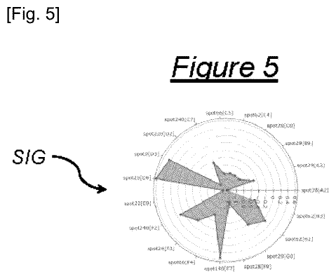

FIG. 5 illustrates, in the form of a pie chart, an example of an olfactory signature that can be computed by the device shown in FIG. 1 from sensorgrams such as those in FIG. 3 or 4,

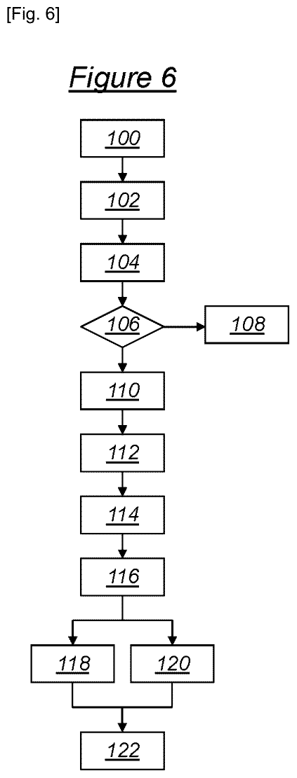

FIG. 6 illustrates the successive steps of a multisensory measurement method for adsorption and desorption of compounds present in a fluid, including automatic temporal labeling by a software module of the electronic device shown in FIG. 1, according to one embodiment of the invention,

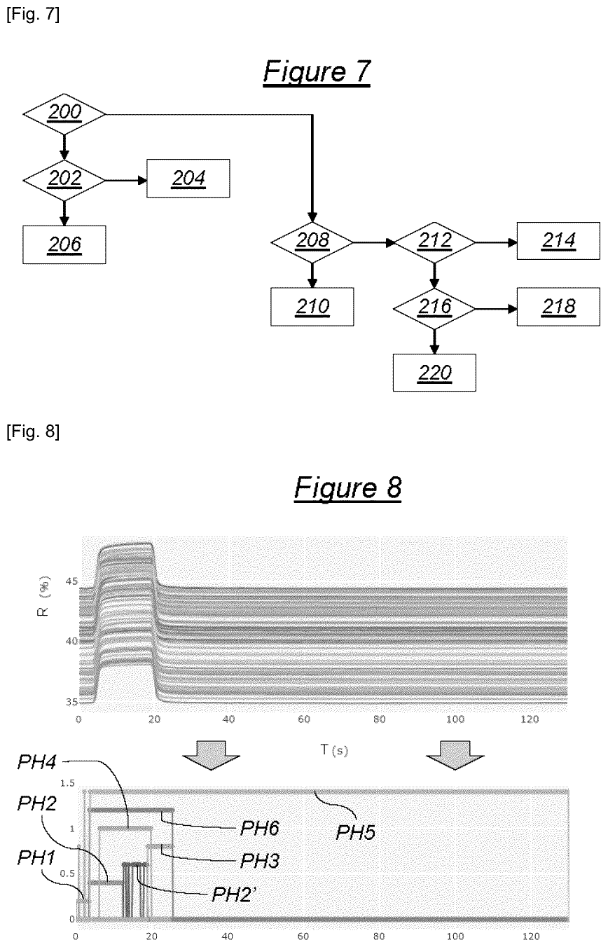

FIG. 7 illustrates the successive steps of a binary signal logical combination method that can be implemented when executing the method shown in FIG. 6,

FIG. 8 illustrates an example of a result that can be obtained by applying the method shown in FIG. 6 to sensorgrams similar to those shown in FIG. 3,

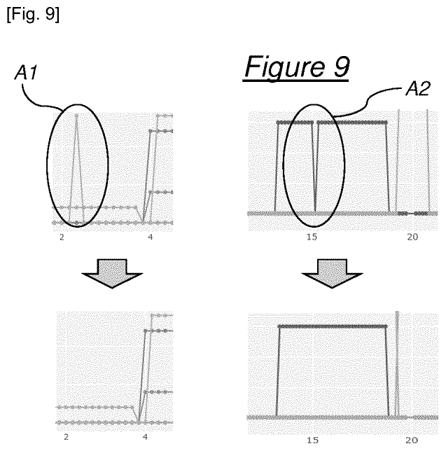

FIG. 9 illustrates a detail of FIG. 8 and an example of a result that can be obtained by applying an optional artifact removal step of the FIG. 6 method,

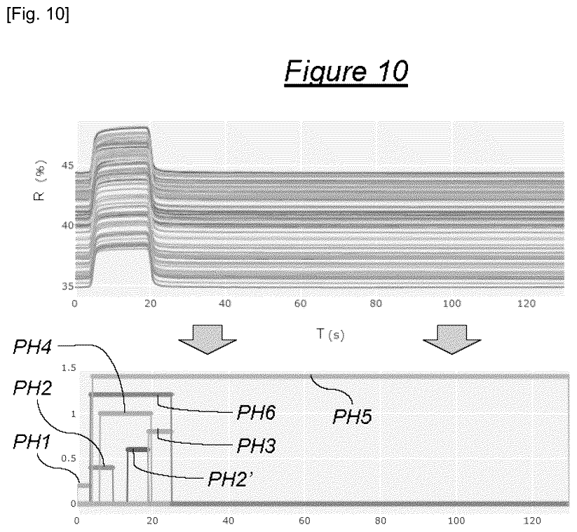

FIG. 10 illustrates another example of a result that can be obtained by applying the method shown in FIG. 6 to sensorgrams similar to those shown in FIG. 3, after execution of the optional artifact removal step,

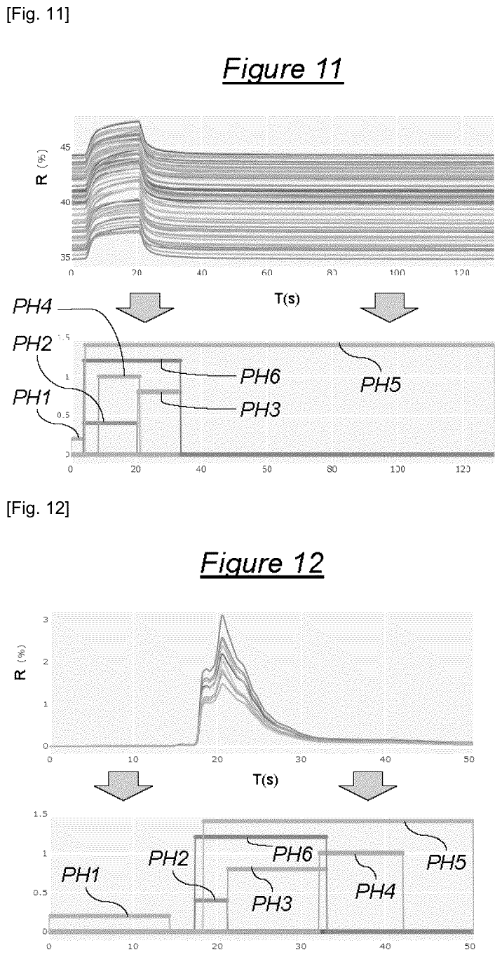

FIG. 11 illustrates an example of a result that can be obtained by applying the method shown in FIG. 6 to the sensorgrams shown in FIG. 3, and

FIG. 12 illustrates an example of a result that can be obtained by applying the method shown in FIG. 6 to the sensorgrams shown in FIG. 4.

The electronic device 10 for multisensory measuring the adsorption and desorption of compounds present in a fluid diagrammatically shown in FIG. 1 is a non-limiting example of a measuring device according to the present invention for a non-limiting odor identification application. It comprises a measuring chamber 12 designed to receive a fluid, for example a gas such as ambient air. To this end, it comprises a suction device 14 designed to draw in air from outside the measuring chamber 12 and bring it inside. It further has an air outlet 16 which can be selectively closed to keep ambient air in the measuring chamber 12, or opened to allow ambient air to be evacuated from the measuring chamber 12 and renewed by activation of the suction device 14. It is thus equipped with means for controlling incoming and outgoing flows.

The device 10 comprises a number of olfactory sensors 18 in its measuring chamber 12, all distributed over as many reactive sites, for example around sixty, designed to interact with volatile organic compounds likely to be present in the measuring chamber 12 when the device 10 is placed close to a fluid to be analyzed which emits these compounds, in particular when the suction device 14 is close to the fluid in question. Each olfactory sensor 18 is, for example, a biosensor designed to interact with compounds from a particular family of volatile organic compounds. In practice, each olfactory sensor 18 may comprise a molecule, such as a peptide immobilized on a substrate or a polymer covering a surface, complementary to the compounds of the family associated with this olfactory sensor 18.

Alternatively, the device 10 could be adapted to be brought into contact with any fluid, liquid or gaseous, other than ambient air. In a particularly basic version, it could also not include the suction device 14 and the air outlet 16, or even the measuring chamber 12. In this basic version, the olfactory sensors 18 can therefore be brought into direct contact with the fluid to be analyzed, without flow control.

The olfactory sensors 18 are associated with at least one transducer 20 which is arranged and configured to measure any change in physical property caused by interaction with the fluid to be analyzed. The transducer 20 provides an electrical signal S which characterizes this fluid since it is representative of the volatile organic compounds with which the olfactory sensors may interact in the measurement chamber 12.

More specifically, the transducer 20 can be a surface plasmon resonance imaging system, i.e., an SPR (for Surface Plasmonic Resonance) imaging system configured to measure any change in a refractive index due to an interaction of the fluid under study with at least one of the olfactory sensors thanks to a plasmonic effect. Such a transducer comprises: a metal layer, a first face of which with reactive sites serves as a support for the olfactory sensors 18; an optical prism arranged against a second face, opposite the first one, of the metal layer; a device for illuminating this second face of the metal layer with collimated and polarized light via a light input face of the optical prism; and a camera arranged at the light output of the optical prism for providing the signal S in the form of a sequence of grayscale images of the reactive sites on which the olfactory sensors 18 are arranged. The reactive sites on the first side of the metal layer are organized, for example, in a matrix positioning grid.

FIG. 2 illustrates a typical schematic example of an image produced by such an SPR imaging system, wherein the olfactory sensors 18 of the measuring device 10 are visible with their luminance values significant of the volatile organic compounds with which they may have interacted. Note that in the example described, the olfactory sensors 18 are circular. However, because the camera is tilted to a certain extent in relation to their positioning grid, the areas they occupy in the gray-scale image sequence S are ellipses.

Alternatively, the SPR imaging system of transducer 20 could be replaced by an optical index variation amplification system using Mach-Zehnder interferometry. Such a system is configured to measure any change in refractive index due to interaction of the fluid under study with at least one of the olfactory sensors by means of a detectable phase shift between a reference arm of the interferometer and a sensing arm on which any such reactive site is disposed. The resulting transducer provides a signal S in the form of a sequence of images of phase shifts, expressed in Radian, of the olfactory sensors 18.

In another variant, the SPR imaging system forming part of transducer 20 could be replaced by a nano- or micro-electromechanical system (NEMS or MEMS). Such a system is configured to measure any change in the resonant frequency of a vibrating membrane on which any one of the olfactory sensors is placed. The reactive sites on which the olfactory sensors are placed are arranged, for example, in a matrix of NEMS or MEMS vibrating membranes to provide a signal S in the form of a sequence of signals of shifts in the resonant frequencies of the olfactory sensors 18.

Other variants are conceivable by implementing any other equivalent physical transduction device (i.e. optical, mechanical, etc.), with a simple adaptation of the measuring device 10 which will not be described because it is within the reach of the person skilled in the art. Whatever the choice of transducer 20, the general idea remains to functionalize reactive sites using olfactory sensors 18 (i.e. biosensors, polymers, carbon nanotubes, etc.) in such a way that they differentially adsorb and desorb volatile organic compounds, to form a molecular interaction response differentiated from the olfactory sensors, and to amplify the response in the form of an electrical signal S using a physical transduction device.

Returning to FIG. 1, the device 10 further comprises a plurality of functional modules which will be described below. In the example described, these modules are of a software nature. Thus, device 10 comprises a computer-like element 22 having a processing unit 24 and an associated memory area 26 wherein a plurality of computer programs or a plurality of functions of a single computer program are stored. These computer programs comprise instructions designed to be executed by the processing unit 24 in order to realize the functions of the software modules. They are presented as distinct, but this distinction is purely functional. They could just as easily be grouped together in any combination to form one or more software modules. Their functions could also be at least partly micro-programmed or micro-wired into dedicated integrated circuits, such as digital circuits. Alternatively, the computer 22 could be replaced by an electronic device consisting solely of digital circuits (without a computer program) to perform the same actions.

The device 10 thus comprises first of all a software module 28, to be executed by the processing unit 24, for controlling the suction device 14 (if provided in the measuring device 10), the air outlet 16 (if also provided in the measuring device 10) and the transducer 20.

Optionally, but advantageously, it further includes a software module 30, to be executed by the processing unit 24, for selecting, from among the olfactory sensors 18 of the measuring device 10, a subset of sensors sensitive to volatile compounds characteristic of a desired olfactory fingerprint. These characteristic volatile compounds may vary from one application or fluid studied to another, so that the selection of olfactory sensors made by software module 30 may also vary and be parameterized. The selected subset comprises, for example, M≥1 olfactory sensor(s), or advantageously a plurality of olfactory sensors (M≥2).

The device 10 further comprises a software module 32, to be executed by the processing unit 24, for extracting M sensorgrams or, more precisely in the non-limiting example of an SPR imaging system, M reflectance signals respectively representative of the interactions of the M selected olfactory sensors with the relevant volatile organic compounds from the luminance values specific to these M selected olfactory sensors in the image sequence S provided by the camera. These reflectance signals are, for example, expressed as a percentage according to a ratio of luminance values obtained with transversely polarized light to luminance values obtained with the same light polarized at 90 degrees for each of the M selected olfactory sensors.

FIG. 3 illustrates the superimposed temporal diagrams of some forty reflectance signals obtained over a period of approximately 130 seconds according to a rigorous measurement protocol involving control of the suction device 14 and the air outlet 16, wherein:
the reactive sites 18 are first exposed to a carrier fluid reference fluidic environment without the presence of the target compounds of a fluid to be analyzed during a first reference state PH1,
they are then exposed to the fluid to be analyzed during a second analytical adsorption state PH2 triggered by controlled injection of this fluid into the measuring chamber 12, and
they are finally re-exposed to the reference fluidic environment during a third final desorption state PH3 by controlled evacuation of the fluid to be analyzed outside the measuring chamber 12.

In this relatively optimal case, the three successive states can be considered as known a priori, and the reflectance signals obtained are of good quality. The equilibrium steady state is clearly not reached at the end of the second state PH2 in the example shown in FIG. 3, because desorption occurs a little too quickly, but it would have been reached without difficulty with a slightly longer adsorption.

FIG. 4, on the other hand, illustrates the superimposed temporal diagrams of ten or so reflectance signals obtained over a period of around 50 seconds using a much less rigorous measurement protocol, such as “approaching”, i.e., approaching the measuring device 10 to the fluid to be analyzed without controlling the suction device 14 and the air outlet 16. In this less ideal case, the three successive states PH1, PH2, PH3 are not known a priori, the reflectance signals obtained are of poorer quality, and the equilibrium steady state never seems to be reached.

It is therefore an object of the present invention to provide automatic temporal labeling indicating a start and an end of at least one adsorption state and one desorption state relative to the signals provided by the software module 32. The device 10 therefore comprises a software module 34, to be executed by the processing unit 24, for determining the temporal evolution of at least one variable estimated on the basis of a combination of temporal variations of the signals provided by the software module 32 and, using this temporal evolution, providing a temporal labeling indicating a start and an end of at least one adsorption state and one desorption state relative to the provided signals. The operation of this software module 34 will be detailed with reference to FIG. 6.

In a known per se manner, the device 10 further includes a software module 36, to be executed by the processing unit 24, for selecting a temporal window for analyzing the M aforementioned reflectance signals with a view to extracting M components of an olfactory signature SIG representative of the fluid under study. In the state of the art, this selection is made on the basis of a priori knowledge of the aforementioned states PH1, PH2 and PH3. The temporal window is selected, for example, at the end of adsorption PH2 and/or at the start of desorption PH3. In accordance with the present invention, this selection is advantageously made on the basis of the automatic temporal labeling performed by software module 34, for example at the end of the temporally labeled adsorption state and/or at the start of the temporally labeled desorption state, and/or as a function of other possibly temporally labeled states as will be detailed below.

The device 10 further includes a software module 38, to be executed by the processing unit 24, for obtaining the M components of the aforementioned olfactory signature SIG from the M reflectance signals.

An example of a 19-component SIG olfactory signature shown as a pie chart is illustrated in FIG. 5, for example obtained by sequential execution of software modules 28 to 36, on the basis of reflectance signals such as those shown in FIGS. 3 and 4. This sequential execution can also be repeated several times to obtain a plurality of olfactory signatures of the same fluid to be analyzed, which can then be statistically processed, for example by averaging or otherwise, to obtain an improved final olfactory signature.

When transducer 20 is a Mach-Zehnder interferometry system, software module 32 is adapted in a manner known per se to extract M phase shift signals instead of the aforementioned M reflectance signals. These phase shift signals are expressed in Radian, for example. However, these are sensorgrams which can also be used. Software modules 36 and 38 are also simply adapted to the phase-shift signals to select the appropriate temporal window and obtain the olfactory signature.

When transducer 20 is a NEMS or MEMS system, software module 32 is adapted in a manner known per se to extract M resonant frequency shift signals instead of the aforementioned M reflectance signals. These resonance frequency shift signals are expressed in Hz, for example. Nevertheless, these are sensorgrams that can also be used. Software modules 36 and 38 are also simply adapted to the resonance frequency shift signals in order to select the appropriate temporal window and obtain the olfactory signature.

FIG. 6 illustrates the details of a possible operation of the temporal labeling software module 34 when executed by the processing unit 24. In this non-limiting example, it is executed after obtaining M complete sensorgrams in a measurement temporal window extending over N successive samples of each of these M sensorgrams provided by the software module 32 in digital form. These N samples correspond to N successive measurement instants or to the sampling of a continuous electrical signal. This mode of operation can be described as “post-processing”. In this operating mode, it takes more than two minutes to process the sensorgrams of FIG. 3, and just under a minute for those in FIG. 4. It should be noted, however, that a simple adaptation of the operation which will now be detailed, within the reach of the person skilled in the art, enables temporal labeling in “real-time” mode, i.e. according to a parallel execution of the two software modules 32, 34, and even more globally of the software modules 28 to 34, whereby the software module 34 carries out and completes its temporal labeling as and when it receives successive sensorgram samples provided by the software module 32. In this real-time mode, the specific processing linked to the evolutionary labeling can extend over a time scale of the order of a second.

In an optional first filtering step 100, the M sensorgrams are filtered by a low-pass filter implemented as a finite or infinite impulse response digital filter. This filters out the high-frequency measurement noise in the raw signals as provided by software module 32. A Butterworth filter with first-order finite impulse response and cutoff frequency normalized to 0.45 (i.e., value of the ratio between cutoff frequency and sampling frequency equal to 0.45) is suitable. Let us denote SG∈ [symbol] ^{M×N} the matrix formed from the M filtered sensorgrams of sampled temporal length N and SG∈ [symbol] ^{M×N} the matrix obtained from SG by a norm calculation within the meaning of patent document WO 2020/141281 A1. The M row vectors of the SG and SG matrices are the M sensorgrams, respectively filtered and filtered then normalized, of length N, while their N column vectors represent the M values taken by the M sensorgrams, respectively filtered and filtered then normalized, at each of the N successive sampling instants t₁ to t_{N}.

In a subsequent step 102, the matrix of normalized sensorgrams SG is computed. In this same step, a number of signals relating to the temporal evolutions of variables estimated on the basis of a combination of temporal variations of the sensorgrams SG and a combination of temporal variations of the normalized sensorgrams SG are computed. At least one of these signals is advantageously used to provide the aforementioned temporal labeling.

A first signal δ̅∈ [symbol] ^{N} of temporal variation of the matrix SG sensorgrams is a temporal standard deviation computed over a sliding window W and averaged over the M sensorgrams SG. It is represented as a line vector. The temporal window W is, for example, about 0.5 seconds, i.e., about ten samples at a sampling frequency of 20 Hz.

A second signal [symbol] ∈ [symbol] ^{N} of temporal variation of the normalized sensorgrams of the matrix SG is another temporal standard deviation computed over the sliding window W and averaged over the M normalized sensorgrams SG. It is also represented as a line vector. More precisely, it takes its values in the space [0,1]^{N}, so that a complement to 1 of this second signal can also be defined, for example noted

$$ϑ = 1 - \overline{\underline{δ}} ∈ {[ 0 , 1 ]}^{N} .$$

A third signal d∈ [symbol] ^{N} of temporal variation of the matrix SG sensorgrams is an instantaneous first derivative averaged over the M sensorgrams SG. It is also represented as a line vector.

A fourth signal d̅∈ [symbol] ^{N} of temporal variation of the matrix SG sensorgrams is a temporal average of the first derivative d, for example computed over the sliding window W. It is also represented as a line vector.

Then, in a step 104, a maximum standard deviation Δ of K first samples of the matrix SG sensorgrams is computed:

$$Δ = {max}_{1 \leq i \leq K} \overline{δ} ( i ) .$$

For this purpose, it is assumed that the first K samples considered result from exposure of the electronic measuring device 10 to a predetermined reference fluidic environment, for example a stable environment with no fluid to be analyzed present. This predetermined reference fluidic environment is associated with a predetermined maximum standard deviation value MAX_{δ̅ref}. K represents, for example, one to two seconds of the sensorgrams SG, i.e., between twenty and forty samples at 20 Hz. As a result, for an environment to be considered stable and reference, it may be deemed necessary and sufficient for the sensorgrams to exhibit a sufficiently low standard deviation δ̅ (i.e., less than MAX_{δ̅ref})) for a sufficiently long time (i.e., for at least K samples). It appears that there is then no need for a priori knowledge of this reference environment. This is a robust assumption, notably because for the majority of e-nose applications, odorless air is air in an environment that contains no odor source, hence no variable effluents, so it is characterized by a low standard deviation δ̅.

In a subsequent test 106, the value of Δ is compared with MAX_{δ̅ref}. If Δ>MAX_{δ̅ref}, then it is considered that the measurement conditions are poor, for example due to an environment that is too noisy to be considered suitable for use as a reference, so that temporal labeling is terminated (step 108). Otherwise, the method continues with a sequence of steps 110 to 114 for temporal labeling of the first reference state PH1 of exposure of the electronic measuring device 10 to the reference fluidic environment.

Alternatively, particularly when the measuring device 10 is used continuously without any particular control over the reference fluidic environment, temporal labeling can simply be suspended, or put on standby, until a new temporal window of at least K samples can be detected, during which the standard deviation δ̅ remains below MAX_{δ̅ref}. In this way, it is not only clear that there is no need for a priori knowledge of the reference environment, but also that there is no need to force the measuring device 10 to be subjected to such a reference environment right from the start of the measurement.

Step 110 consists in determining the first sample of the sensorgrams of matrix SG, of index i_{ref}≤N, for which δ̅(i)≥α_{δ̅}^{ref}×Δ, where αδ̅^{ref} is a predefined tolerance coefficient, advantageously strictly greater than 1, for example equal to 1.1.

In the next step 112, the first reference state PH1 is temporally labeled by a binary signal F_{PH1} of temporal length N defined as follows:

$$\{ \begin{matrix}∀ i , 1 \leq i \leq {i}_{ref} , {F}_{PH 1} ( i ) = 1 \\ ∀ i , {i}_{ref} < i \leq N , {F}_{PH 1} ( i ) = 0\end{matrix} .$$

In other words, the reference state PH1 extends from sample 1 to sample i_{ref}, i.e. as long as the binary signal F_{PH1} is 1. According to the aforementioned variant of continuous use of the measuring device 10, the first sample of the reference state PH1 is the first of the new temporal window of at least K successive samples that can be considered as coming from a sufficiently stable environment in the sense of δ̅ to constitute a reference.

In the next step 114, optional initializations are proposed for further temporal labeling, based on the already labeled reference state PH1.

A first optional initialization consists of computing an offset signal O∈ [symbol] ^{M} of the reference state PH1. This signal O is represented as a column vector and computed as follows:

$$∀ m ∈ [ 1 , M ] , O ( m ) = \frac{1}{{i}_{ref}} {\sum }_{i = 1}^{{i}_{ref}} SG ( m , i ) .$$

This offset signal is then subtracted from each of the column vectors of the matrix SG.

A second initialization, also optional, consists of defining a plurality of threshold values.

A first value is a threshold for the standard deviation δ̅ related to the sensorgrams SG, aimed at detecting the adsorption state PH2 and the desorption state PH3. This threshold ρ_{δ̅} is for example defined in the following adaptive way:

$${ρ}_{\overline{δ}} = {max}_{1 \leq i \leq {i}_{ref}} ( \overline{δ} ( i ) ) .$$

A second value is a threshold for the temporal-averaged first derivative d̅ related to the sensorgrams SG, aimed at detecting the adsorption state PH2. This threshold ρ_{d̅}^{ad} is for example defined in the following adaptive way:

$${ρ}_{\overline{d}}^{ad} = {max}_{1 \leq i \leq {i}_{ref}} ( \overline{d} ( i ) ) .$$

A third value is a threshold for the temporal-averaged first derivative d̅ related to the sensorgrams SG, aimed at detecting the desorption state PH3. This threshold ρ_{d̅}^{des} is for example defined in the following adaptive way:

$${ρ}_{\overline{d}}^{des} = {max}_{1 \leq i \leq {i}_{ref}} ( \overline{d} ( i ) ) .$$

A fourth value is a threshold for the standard deviation [symbol] related to the normalized sensorgrams SG, optionally aimed at detecting a state of higher stability PH4. This threshold

$${ρ}_{\underline{δ}}^{\underline{s} table}$$

is for example defined in the following adaptive way:

$${ρ}_{\overline{\underline{δ}}}^{stable} = {max}_{1 \leq i \leq N} ( \overline{\underline{δ}} ( i ) ) .$$

A fifth value is a threshold for the standard deviation [symbol] related to the normalized sensorgrams SG, optionally aimed at detecting an odor presence state PH5. This threshold

$${ρ}_{\underline{δ}}^{\underline{o} d}$$

is for example defined in the following adaptive way:

$${ρ}_{\overline{\underline{δ}}}^{od} = {max}_{1 \leq i \leq {i}_{ref}} ( \overline{\underline{δ}} ( i ) ) .$$

In addition, it is advantageous to define a plurality of tolerance coefficients relating to at least some of the aforementioned thresholds. For example, a tolerance coefficient α_{δ̅} as applies to the threshold ρ_{δ̅} to label the adsorption and desorption states PH2, PH3; a tolerance coefficient α_{δ̅}^{p} applies to the threshold ρ_{δ̅} to label an optional plateau state PH2′; a tolerance coefficient α_{d̅}^{ad} applies to the threshold ρ_{d̅}^{ad} pad to label the adsorption state PH2; a tolerance coefficient α_{d̅}^{des} applies to the threshold ρ_{d̅}^{des} to label the desorption state PH3; a tolerance coefficient

$${α}_{\underline{δ}}^{\underline{s} table}$$

applies to the threshold

$${ρ}_{\underline{δ}}^{\underline{s} table}$$

to label the state of higher stability PH4; a tolerance coefficient

$${α}_{\underline{δ}}^{\underline{o} d}$$

applies to the threshold

$${ρ}_{\underline{δ}}^{\underline{o} d}$$

to label the odor presence state PH5. All these predefined tolerance coefficients are advantageously strictly greater than 1, for example equal to 1.1, with the exception of the tolerance coefficient α_{δ̅}^{p} which is advantageously strictly less than 1, for example equal to 0.9.

Alternatively, the five above-mentioned threshold values can be independently defined by default and predetermined, in which case it is not necessary to define tolerance coefficients.

Then, in a step 116, a number of binary signals of temporal length N are defined for labeling states PH2, PH2′, PH3, PH4 and PH5.

According to a non-limiting example, a first intermediate binary signal F_{δ̅} of combined labeling of adsorption and desorption states PH2, PH3 is defined as follows from the temporal standard deviation δ̅:

$$\{ \begin{matrix}∀ i , 1 \leq i \leq N , {F}_{\overline{δ}} ( i ) = 1 if \overline{δ} ( i ) > {α}_{\overline{δ}} \times {ρ}_{\overline{δ}} \\ ∀ i , 1 \leq i \leq N , {F}_{\overline{δ}} ( i ) = 0 if \overline{δ} ( i ) \leq {α}_{\overline{δ}} \times {ρ}_{\overline{δ}}\end{matrix} .$$

A second intermediate binary signal Fad of specific labeling of the adsorption state PH2 is defined as follows from the temporal-averaged first derivative d:

$$\{ \begin{matrix}∀ i , 1 \leq i \leq N , {F}_{\overline{d}}^{ad} ( i ) = 1 if \overline{d} ( i ) > {α}_{\overline{d}}^{ad} \times {ρ}_{\overline{d}}^{ad} \\ ∀ i , 1 \leq i \leq N , {F}_{\overline{d}}^{ad} ( i ) = 0 if \overline{d} ( i ) \leq {α}_{\overline{d}}^{ad} \times {ρ}_{\overline{d}}^{ad}\end{matrix} .$$

A third intermediate binary signal F_{d̅}^{des} of specific labeling of the desorption state PH3 is defined as follows from the temporal-averaged first derivative d:

$$\{ \begin{matrix}∀ i , 1 \leq i \leq N , {F}_{\overline{d}}^{des} ( i ) = 1 if \overline{d} ( i ) < {α}_{\overline{d}}^{des} \times {ρ}_{\overline{d}}^{des} \\ ∀ i , 1 \leq i \leq N , {F}_{\overline{d}}^{des} ( i ) = 0 if \overline{d} ( i ) \geq {α}_{\overline{d}}^{des} \times {ρ}_{\overline{d}}^{des}\end{matrix} .$$

As for the optional plateau state PH2′, it can be temporally labeled directly by a binary signal F_{PH2}, of temporal length N defined as follows from the temporal standard deviation δ̅:

$$\{ \begin{matrix}∀ i , 1 \leq i \leq N , {F}_{PH 2 ′} ( i ) = 1 if \overline{δ} ( i ) \leq {α}_{\overline{δ}}^{p} \times {ρ}_{\overline{δ}} \\ ∀ i , 1 \leq i \leq N , {F}_{PH 2 ′} ( i ) = 0 if \overline{δ} ( i ) > {α}_{\overline{δ}}^{p} \times {ρ}_{\overline{δ}}\end{matrix} .$$

In other words, the plateau state PH2′ extends as long as the binary signal F_{PH2′} is 1.

The state of higher stability PH4 can be temporally labeled directly by a binary signal F_{PH4} of temporal length N defined as follows from the complement to 1 ν of the normalized temporal standard deviation [symbol] :

$$\{ \begin{matrix}∀ i , 1 \leq i \leq N , {F}_{PH 4} ( i ) = 1 if ϑ ( i ) > {α}_{\overline{\underline{δ}}}^{stable} \times {ρ}_{\overline{\underline{δ}}}^{stable} \\ ∀ i , 1 \leq i \leq N , {F}_{PH 4} ( i ) = 1 if ϑ ( i ) \leq {α}_{\overline{\underline{δ}}}^{stable} \times {ρ}_{\overline{\underline{δ}}}^{stable}\end{matrix} .$$

In other words, the state of higher stability PH4 extends as long as the binary signal F_{PH4} is 1.

Finally, the odor presence state PH5 can be temporally labeled directly by a binary signal F_{PH5} of temporal length N defined as follows from the complement to 1 ν of the normalized temporal standard deviation [symbol] :

$$\{ \begin{matrix}∀ i , 1 \leq i \leq N , {F}_{PH 5} ( i ) = 1 if ϑ ( i ) > {α}_{\overline{\underline{δ}}}^{od} \times {ρ}_{\overline{\underline{δ}}}^{od} \\ ∀ i , 1 \leq i \leq N , {F}_{PH 5} ( i ) = 1 if ϑ ( i ) \leq {α}_{\overline{\underline{δ}}}^{od} \times {ρ}_{\overline{\underline{δ}}}^{od}\end{matrix} .$$

In other words, the odor presence state PH5 continues as long as the binary signal F_{PH5} is 1.

In a subsequent step 118, the adsorption state PH2 can be temporally labeled by a binary signal F_{PH2} of temporal length N resulting from a logical combination of the first and second intermediate binary signals F_{δ̅} and F_{d̅}^{ad}. A logic combination method such as that shown in FIG. 7 can for example be used for this purpose.

According to a first test step 200 of this logic combination method, it is verified that neither of the two intermediate binary signals F_{δ̅} and F_{d̅}^{ad} is zero over the entire sampling time. If this is the case, the logic combination method proceeds to another test step 202.

The test step 202 consists in verifying that the first intermediate binary signal F_{δ̅} does indeed have two distinct temporal states at 1, one then relating to the adsorption state PH2, the other one to the desorption state PH3. If this is not the case, the logic combination method proceeds to a final labeling step 204. If it does, it proceeds to another final labeling step 206.

The final labeling step 204 consists in disregarding the first intermediate binary signal F_{δ̅} and defining the binary signal F_{PH2} as equal to the second intermediate binary signal F_{d̅}^{ad}. On the contrary, the final labeling step 206 consists in taking into account the first intermediate binary signal F_{δ̅} and defining the binary signal F_{PH2} as being equal to the logical operation F_{δ̅} AND F_{d̅}^{ad}, i.e., the logical AND operation performed bit by bit on these two signals.

If the result of step 200 is that at least one of the two intermediate binary signals F_{δ̅} and F_{d̅}^{ad} is zero over the entire sampling duration, then the logic combination method proceeds to a test step 208 wherein it is checked whether the two intermediate binary signals F_{δ̅} and F_{d̅}^{ad} are zero over the entire sampling duration.

If true, this is followed by a step 210 to terminate temporal labelling (or standby). This step is similar to step 108 in that it is assumed that the measurement conditions do not allow adsorption and desorption states to be correctly identified. All other binary temporal labeling signals are set to 0 during this step 210.

If one of the two intermediate binary signals F_{δ̅} et F_{d̅}^{ad} is not zero over the entire sampling duration, a new test step 212 is performed, wherein it is checked whether it is the intermediate binary signal F_{δ̅} that is zero over the entire sampling duration. If this is the case, the logic combination method proceeds to a final labeling step 214 identical to step 204: F_{PH2}=F_{d̅}^{ad}. Otherwise, the intermediate binary signal Fad is zero over the entire sampling duration, and the method proceeds to a new test step 216 identical to step 202.

This step 216 therefore consists in checking that the first intermediate binary signal F_{δ̅} does indeed have two distinct temporal states at 1, one then relating to the adsorption state PH2, the other to the desorption state PH3. If this is not the case, the logic combination method moves on to an end of temporal labeling step 218 identical to step 210. If it does, it proceeds to a final labeling step 220.

The final labeling step 220 consists in taking into account only the first intermediate binary signal F_{δ̅} to define the binary signal F_{PH2} as being equal to F_{δ̅} until the end of its first temporal state at 1, the one which is precisely considered as relating to the adsorption state PH2.

Regardless the final labeling step 204, 206, 214 or 220 with which the logic combination method of FIG. 7 ends, the adsorption state PH2 obtained in this way in step 118 of the temporal labeling method of FIG. 6 extends as long as the binary signal F_{PH2} is 1.

Following step 116 of the temporal labeling method in FIG. 6, the desorption state PH3 can be temporally labeled in a step 120 by a binary signal F_{PH3} of temporal length N resulting from a logical combination of the first and third intermediate binary signals F_{δ̅} and F_{d̅}^{des}. The logic combination method of FIG. 7 can also be applied for this purpose by replacing the intermediate binary signal F_{d̅}^{ad} with F_{d̅}^{des}. Thus, applied to the signals F_{δ̅} and F_{d̅}^{des}, the final labeling step 204 provides the binary signal F_{PH3}=F_{d̅}^{des}, the final labeling step 206 provides the binary signal F_{PH3}=F_{d̅}^{des} and the final labeling step 220 provides the binary signal F_{PH3} by defining it as being equal to F_{δ̅} from the start of its second temporal state at 1, the one precisely considered as relating to the desorption state PH3, but zero before it.

At the end of steps 116, 118 and 120, the six previously defined states PH1, PH2, PH2′, PH3, PH4 and PH5 are temporally labeled by their respective binary signals F_{PH1}, F_{PH2}, F_{PH21}, F_{PH3}, F_{PH4} et FPHS. A seventh state PH6, which can be described as an odorant state, can be defined as starting with the adsorption state PH2 and ending with the desorption state PH3. It is accordingly labeled by a binary signal F_{PH6}.

The lower section of FIG. 8 illustrates the results that can be obtained from sensorgrams similar to those shown in FIG. 3, i.e., according to a rigorously controlled measurement protocol. These signals are visible in the upper part of FIG. 8, and it is clear that the equilibrium steady state is reached before desorption, in contrast to the example in FIG. 3. The high binary values of the different states are themselves differentiated on the abscissa in the lower temporal diagram of FIG. 8 to distinguish the different states more clearly, but this choice is only of illustrative interest without being restrictive: the high binary value is, for example, 0.2 for state PH1, 0.4 for state PH2, 0.6 for state PH2′, 0.8 for state PH3, 1 for state PH4, 1.5 for state PH5 and 1.25 for state PH6. The above seven states are all temporally labeled.

It should be noted that although the plateau state PH2′ and the higher stability state PH4 are correlated, they are nevertheless labeled quite differently because they are not evaluated on the same signals: the signals SG for the plateau state PH2′ and the normalized signals SG for the higher stability state PH4. They are therefore complementary. Since the plateau state PH2′ is included in the higher stability state PH4, we can deduce that its detection is more demanding.

It should also be noted that although the odor presence PH5 and odorant PH6 states are correlated, they are nevertheless labeled in rather different ways because they are not evaluated on the same signals either: the signals SG for the odorant state PH6 and the normalized signals SG for the odor presence state PH5. They therefore also show a certain degree of complementarity. However, they start at roughly the same time, at the start of the adsorption state.

Finally, we note the presence of a few artifacts. The labels of states PH3 and PH5 occasionally and accidentally take on their high binary values during the first reference state PH1, while the plateau state PH2′ is visibly unstable.

Following steps 116, 118 and 120, an optional final step 122 can be proposed to remove any artifacts. These artifacts are due either to jitter in the temporal labeling binary signals outside the corresponding active states, or to discontinuities in these signals in the active states they are intended to label. Two threshold parameters are therefore defined for this purpose, one for a minimum threshold ζₘᵢₙ to impose the minimum length of an active state in a labeling binary signal of this state (i.e., elimination of high binary value jitter), the other for a maximum threshold ζₘₐₓ to impose the maximum length of a discontinuity within an active state in a labeling binary signal of this state (i.e., elimination of low binary value discontinuities). Thus, on all the temporal labeling signals F_{PH1}, F_{PH2}, F_{PH2′}, F_{PH3}, F_{PH4}, F_{PH5} and F_{PH6} obtained in steps 116, 118 and 120, these two thresholds Imin and Imax are applied successively to, on the one hand, retain only active states with lengths greater than or equal to ζₘᵢₙ and, on the other hand, reset discontinuities with lengths less than or equal to ζₘₐₓ to high binary values. Essentially, this procedure eliminates any artifacts that may be present.

FIG. 9 shows a magnified detail of temporal labeling signals before (top) and after (bottom) artifact removal, for example for values of ζₘᵢₙ and ζₘₐₓ equal to five samples. A first artifact A1 consists of a jitter in a first temporal labeling binary signal. It is eliminated because this jitter does not exceed the minimum length ζₘᵢₙ. A second artifact consists of a discontinuity in the active state of a second temporal labeling binary signal. It is eliminated because it does not reach the maximum length ζₘₐₓ.

FIG. 10 accordingly illustrates the result that can be obtained after performing step 122 on the example shown in FIG. 8. All seven of the above-mentioned states are temporally labeled, and there are no more artifacts.

FIG. 11 illustrates the result that can be obtained by executing the temporal labeling method of FIG. 6 on the sensorgrams of FIG. 3. Note that the plateau state PH2′ is not detected and therefore labeled.

FIG. 12 illustrates the result that can be obtained by executing the temporal labeling method of FIG. 6 on the sensorgrams of FIG. 4. It should also be noted that the plateau state PH2′ is not detected and therefore not labeled. However, despite much less rigorous and controlled measurement conditions than in the previous example, it should be noted that six of the seven predefined states are identified and labeled, which testifies to the ability to characterize the fluid to be analyzed in spite of everything.

It clearly appears that a multisensory measurement electronic device such as the one described above can facilitate the characterization of a fluid by its ability to temporally label at least adsorption and desorption states without requiring prior control of successive exposures of the device to a reference fluid and to the fluid to be analyzed. By cleverly studying at least one combination of temporal variations in the sensorgrams, whether these variations are standard deviations and/or first derivatives and/or others, this labeling can be carried out automatically and without a priori knowledge. On-demand or continuous operation is then possible. Moreover, fluid characterization is easier when based on such labeling, because it facilitates the choice of the time window for extracting its olfactory signature.

It should also be noted that the invention is not limited to the embodiment described above. It is particularly complete and efficient, but simpler variants are also possible.

In particular, as an alternative, the temporal labeling of adsorption and desorption states PH2, PH3 could be done either on the basis of the first intermediate binary signal F_{δ̅}, only, or on the basis of the second and third intermediate signals F_{d̅}^{ad} and F_{d̅}^{des}, only, without any logical combination of these signals. Also alternatively, the second and third intermediate signals F_{d̅}^{ad} et F_{d̅}^{des} could be defined from the instantaneous first derivative d instead of the temporal average of this instantaneous first derivative d. Furthermore, it should be noted that automatic temporal labeling of states PH1, PH2′, PH4, PH5 and PH6 is optional.

Alternatively also, the variable estimated on the basis of a combination of temporal variations of the signals provided is not necessarily a standard deviation or a first derivative. It may be another statistical variable or an n-th derivative, n≥2.

More generally, it will be apparent to the person skilled in the art that various amendments can be made to the embodiment described above, in the light of the teaching just disclosed. In the foregoing detailed presentation of the invention, the terms used are not to be construed as limiting the invention to the embodiment set forth in the present description, but are to be interpreted as including all equivalents the anticipation of which is within the reach of the person skilled in the art by applying their general knowledge to the implementation of the teaching just disclosed to them.

1. A multisensory measurement electronic device for multisensory measuring the adsorption and desorption of compounds present in a fluid, comprising:
at least one olfactory sensor configured to:
interact with a plurality of organic compounds likely to be present in the fluid,
provide a plurality of signals representative of a presence of said organic compounds in the fluid; and
a processor for processing configured to process the provided signals to obtain a characterization of the fluid composition;
wherein the processor is programmed to:
determine a temporal evolution of at least one variable estimated on the basis of a combination of temporal variations of the provided signals; and
using said temporal evolution, provide a temporal labeling indicating at least:
a start and an end of an adsorption state, and
a start and an end of a desorption state,
related to the provided signals.

2. The multisensory measurement electronic device according to claim 1, wherein the processor is programmed to:
provide the temporal labeling by comparing said at least one estimated variable to at least one threshold; and
compute said at least one threshold on the basis of temporal variations in the provided signals during a reference state of exposure of said at least one olfactory sensor to a reference fluidic environment.

3. The multisensory measurement electronic device according to claim 1, wherein said at least one estimated variable comprises at least one of the elements of the set consisting of:
a temporal standard deviation computed over a sliding window and averaged over the provided signals;
an instantaneous first derivative averaged over the provided signals; and
a temporally averaged first derivative computed over the sliding window and averaged over the provided signals.

4. The multisensory measurement electronic device according to claim 1, wherein the provided signals comprise at least one of the elements of the set consisting of:
a first set of temporal signals, forming sensorgrams, characteristic of an interaction kinetics between the fluid compounds and said at least one olfactory sensor; and
a second set of normalized temporal signals, forming normalized sensorgrams, obtained from the sensorgrams of the first set and by computing at each measurement or sampling instant a norm of the vector formed by the values assumed by the sensorgrams of the first set at that instant.

5. A multisensory measurement method of adsorption and desorption of compounds present in a fluid, using an electronic device comprising:
at least one olfactory sensor configured to:
interact with a plurality of organic compounds likely to be present in the fluid,
provide a plurality of signals representative of a presence of said organic compounds in the fluid; and
a processor for processing the provided signals to obtain a characterization of the fluid composition;
the method being carried out by execution of the processor and comprising:
determining a temporal evolution of at least one variable estimated on the basis of a combination of temporal variations of the provided signals;
using said temporal evolution, providing a temporal label indicating at least:
a start and an end of an adsorption state, and
a start and an end of a desorption state,
related to the provided signals.

6. The multisensory measurement method according to claim 5, comprising the provision of a temporal labeling of a reference state of exposure of said at least one olfactory sensor to a reference fluidic environment not containing the compounds whose adsorption and desorption are to be measured, said temporal labeling being defined as follows:
start of reference state at first sample of provided signals; and
end of reference state as soon as a temporal standard deviation computed over a sliding window and averaged over the provided signals is greater than or equal to a reference threshold predefined as being proportional to a maximum reached by said temporal standard deviation over K first samples of the provided signals, with K∈ [symbol] ⁺.

7. The multisensory measurement method according to claim 6, wherein the temporal labeling of adsorption and desorption states is defined:
for the adsorption state: by a temporal labeling binary signal indicating the adsorption state by a high binary value, said binary signal resulting from a logical combination:
of a first intermediate binary signal F_{δ̅} with a high binary value for any sample of the provided signals for which the temporal standard deviation computed over a sliding window and averaged over the provided signals is greater than an adsorption/desorption threshold predefined as being proportional to a maximum reached by said temporal standard deviation during the reference state, and
of a second intermediate binary signal F_{d̅}^{ad} with a high binary value for any sample of the provided signals for which a first derivative averaged over the provided signals is greater than an adsorption threshold predefined as being proportional to a maximum reached by said first derivative during the reference state;
for the desorption state: by a temporal labeling binary signal indicating the desorption state by a high binary value, said binary signal resulting from a logical combination:
of the first intermediate binary signal F_{δ̅}, and
of a third intermediate binary signal F_{d̅}^{des} with a high binary value for any sample of the provided signals for which the first derivative averaged over the provided signals is lower than a desorption threshold predefined as being proportional to a minimum reached by said first derivative during the reference state;
and wherein the temporal standard deviation and the first derivative are computed on temporal signals, forming sensorgrams, characteristic of interaction kinetics between fluid compounds and said at least one olfactory sensor.

8. The multisensory measurement method according to claim 7, comprising the provision of a temporal labeling of a plateau state defined by a temporal labeling binary signal indicating the plateau state by a high binary value, said binary signal being with a high binary value for any sample of the provided signals for which the temporal standard deviation computed over the sliding window and averaged over the provided signals is less than or equal to a plateau threshold predefined as being proportional to a maximum reached by said temporal standard deviation during the reference state.

9. The multisensory measurement method according to claim 6, comprising the provision of a temporal labeling of states of higher stability and odor presence defined:
for the state of higher stability: by a temporal labeling binary signal indicating the state of higher stability by a high binary value, said binary signal being of high binary value for any sample of the provided signals for which a complement to 1 of a normalized temporal standard deviation computed over the sliding window and averaged over the provided signals is greater than a threshold of higher stability predefined as being proportional to a maximum reached by said normalized temporal standard deviation during the whole duration of the provided signals; and
for the state of odor presence: by a temporal labeling binary signal indicating the odor presence state by a high binary value, said binary signal being of high binary value for any sample of the provided signals for which the complement to 1 of the normalized temporal standard deviation computed over the sliding window and averaged over the provided signals is greater than an odor presence threshold predefined as being proportional to a maximum reached by said normalized temporal standard deviation during the reference state;
and wherein the normalized temporal standard deviation is computed on normalized temporal signals, forming normalized sensorgrams, obtained from non-normalized temporal signals characteristic of interaction kinetics between fluid compounds and said at least one olfactory sensor and by computing at each measurement or sampling instant a norm of the vector formed by the values assumed by the non-normalized temporal signals.

10. A non-transitory computer readable medium comprising instructions for executing the steps of a multisensory measurement method according to claim 5, when said instructions are executed on a computer capable of processing signals representative of a presence of organic compounds in a fluid, provided by at least one olfactory sensor, to obtain a characterization of the fluid composition.
